(19) United States
(12) Patent Application Publication
(10) Pub. No.: US 2010/0136634 A1
(43) Pub. Date: Jun. 3, 2010

(54) METHOD AND EQUIPMENT FOR PRODUCTION OF GLUCOSE, ETHANOL,FURFURAL,FURANE AND LIGNIN FROM RENEWABLE RAW MATERIALS

(76) Inventors: Zdenek Kratochvil, Praha (CZ); Libor Rychtár, Ostrava-Poruba (CZ); Frantisek Machek, Praha (CZ); Frantisek Bouska, Praha (CZ)

Correspondence Address: DYKEMA GOSSETT PLLC, FRANKLIN SQUARE, THIRD FLOOR WEST, 1300 I STREET, NW, WASHINGTON, DC, 20005, US

(21) Appl. No.: 12/452,944

(22) PCT Filed: Jul. 30, 2007
(86) PCT No.: PCT/CZ2007/000076

§ 371 Date: Jan. 29, 2010

(51) Int. Cl. C12P 19/02; C12P 7/06; C12P 7/04; C12P 7/54; C12P 7/40; C07G 1/00; C12M 1/00

(52) U.S. Cl. 435/105; 435/161; 435/126; 435/157; 435/140; 435/136; 530/500; 435/289.1

(57) ABSTRACT

Method and equipment for production of fermentable saccharides, ethanol, furfural, furane, lignin, acetic acid and formic acid from lignocellulosic and amylaceous materials. The method comprises one-stage or two-stage continuous thermo-compressive hydrolysis of ligno-cellulosic particles, cellulase treatment of unreacted lignocellulose, amylase treatment of formed monosaccharides combined with added amylaceous materials, and fermentation of the combined processed monosaccharide solutions into ethanol. Side products furfural, methanol, acetic acid, formic acid and lignin are recovered and purified, optionally furfural is further converted to furan. An integrated process for recovery and recycling of all products and by-products, and recycling of heat energy is disclosed.

### FIELD OF THE INVENTION

Invention is solving the complex method of working up of ligno-cellulosic and amylaceous materials onto the monosaccharides, glucose, fuel alcohol, furfural, furane, acetic-acid, formic-acid and lignin. According to the origin and composition of ligno-cellulosic materials, for example the proportion of hemicellulose and cellulose itself can be possible to prepare the conditions of thermo-compressive hydrolysis as optimum. The created monosaccharides are the basic energetic raw material for the fermenting preparation of ethanol, lactic-acid and next fermented products.

### BACKGROUND OF THE INVENTION

The lack of fosile raw material sources is becoming as the potential obstacle which can break economical and social development of the most countries. Todays systems of the organic chemical productions are mostly based on the fosile raw materials and non-biologic technologies. Fosile raw materials, namely the oil and earth gas are step by step being exhausted.

Beside the fosile sources of raw materials there is the renewable organic mass for disposal nowadays and namely in future. Organic (biologic) mass further LCM (ligno-cellulosic materials) is the mostly used renewable source of energy now, and it has huge part in all over the world production. From the quality point of view there is the situation of LCM usage completely unsatisfactory. Namely in the economically less developed countries is the wood main material for heating, and the way of wood combustion is uneconomical. The usage of LCM materials from the agricultural production is not on demanded and possible level.

Main producers of LCM materials are the prior sources of agricultural production (various kinds of straw), and the waste from wood and forest industry. If these sources shall be divided into the use in three stages, their usage will be as follows:
in first stage the sources from agricultural production shall be used
in second stage the sources from wood industry shall be used
in third stage the waste materials from forests shall be used

The mostly advanced stage is the verification of LCM materials from primary agricultural production, by which there were reached very good technologic and economic results.

Several following paragraphs are describing certain hydrolytic and dehydratation methods:

The company Quaker Oats used the method of discontinous hydrolysis of ligno-celluloses with sulphur-acid (5% water solution) for production of furaldehyd, with the help of temperature 145 till 170° C.

In the firm Agrifuran there is using the water extract of superphosphate containing 45 wt. % P₂O₅, which is added directly into autoclave.

Sweden company Defibrator designed the continual hydrolysis. They used single-stage expansion, and the raw material is impregnated by sulphur-acid before the hydrolysis.

There is known a CZ patent No. 191945 which is solving the problem with the help of continual two-stage hydrolysis, and the added sulphur-acid. At first stage the hydrolysis is carried out in temperature 150 till 200° C. with concentration of sulphur-acid higher than 10 wt. %. At second stage the same temperature is used, and the concentration of sulphur-acid is recommended up to 5 wt. %.

All above mentioned methods of fural production have the common imperfection. It is comparatively small amount of final product, maximum 30 till 45%, and insufficient upvaluation some parts of used raw materials, namely of rest phase of ligno-cellulosis.

When the fluidic method of furaldehyd production is used, the lowering of final product amount is caused by thermo-oxidative decomposition of furaldehyd in the reaction with air oxygen.

A Swiss patent No. CH 678183 A5 has introduced the acidic hydrolysis of raw materials which contain the pentosans, with the help of sulphur-acid in concentration 2 wt. %, and temperature of 170 till 230° C.

The pentose fraction is utilized all over the world for fural production. Older attempts leading to fural production were based on the pressure and heat applied onto the raw material. Original patents La Forge used the dehydratic reactions with the help of certain organic acid, which was released from the vegetable raw material by the action of superheated vapour (CH₃COOH, HCOOH). The company Quaker Oats started to use sulphur-acid (5% water solution), and the temperature 145 till 147° C.

Beside this technology there is several continuous technologies, which can be divided as follows:

a) direct (single-stage)
b) indirect (two-stage and more-stage).

Indirect methods are two-stage methods, in first stage the solution of saccharoses (with the help of prehydrolysis, hydrolysis, delignification or extraction) has been prepared, and this solution is working up in next stage with the help of dehydratation reaction. There are methods by which the fural in first step from the relief gases can be separating, but most amount of fural is created in second stage of prehydrolysis dehydratation.

During above mentioned production methods of furfural is very hard to prevent the reactions of furfural with other parts creating from this process, and other degradation processes. The creation of furfural and its drawing off from the reaction zone is determined by its diffusion from vegetable materials, in which is creating. There is not possible to remove the air oxygen from the reaction space, what is the main disadvantage of direct methods. Air oxygen presence can cause as much as 10% lowering of fural production.

According to the method of vegetable raw material usage, these methods can be categorized:
fural and hexosis hydrolysate (this variant needs the highest demands for process technology from the point of view of reaction kinetics, temperature and pressure, and so on)
fural and cellulose (fibrous materials)
fural and binding agent, carbon (activated carbon)
fural and fertilizer

Next, there are announced some of used fural production methods (eventually together with other products):

Company Quaker Oats is using the discontinual charging of ligno-cellulosic materials into the autoclave, where 5% sulphur-acid is feeding as well, and the lowest hydromodule is kept, it means as much as 0.5. Rest from hydrolysis is drying at dryer, and it is used as solid fuel or fertilizer. The neutralization of this rest is carrying out by the ammonia.

The firm Thermodynamik has developed similar method of fural production as Quaker Oats, which uses the wood from leaf trees. Water extract of superphosphate (containing 45% of P₂O₅) as additive into autoclave, can be used instead of sulphur-acid (method of Agrifuran).

According to the company Defibrator, the wood chips shall be exposed by the high pressure vapour in continual pressure equipment. The raw material shall be drawn up from the autoclave to the expander, where the pressure shall be lowered to normal atmospheric pressure. There shall be separated fural and water vapour from solid rest which shall be taken away from the expander by the help of endless screw.

Common imperfection of above mentioned methods is the shortage of process efficiency which cannot reach 45% of theoretic calculation, and insufficient valorization of other parts (namely cellulose). In the fluid layer there occurs the degradation decomposition of fural when it is contacting with oxygen. The other difficult reaction is creation of glycosans.

Causes of the breaking down of 2-furaldahyd (furfural) production all over the world can be characterized on the base of reasonable informations as follows:
breaking down of machinery (not very often cases, for example the continual feeder was broken sometimes)
breaking down of technology (it is resulted in very complicated mechanism of 2-furaldehyd creation and decomposition with many following reactions, which are complicated by diffusion reactions in entering raw material, and the specific hydrodynamics of water vapour in the reaction equipment)
low (or zero) profit of production resulting mainly in the usage of unsuitable technology for the given ligno-cellulosic material, its production capacity and the energetic demands.

There shall be necessary to develop or to invent cheaper and more effective methods of raw material conversion to the ecologic gas and fluid fuels at near future, which shall make possible wider usage of biomass and renewable sources in whole, i.e. without limitation of the raw material source distance, and also higher flexibility for methods of their applications and without seasons vicissitudes.

As mentioned above, the methods for furaldehyd (fural) production, further the lignin and hydrolytic saccharides production. There has been concentrated attention to ethanol production based on the amylaceous and ligno-cellulosic materials.

In spite of the fact that nowadays' practice of usage of the fosile raw material for ethanol production is more effective, the greatest petrochemistry companies found their research and development workshops for investigation of new technologies based on renewable sources. The conference European on Bioethanol was engaged in the causes of the unfavourable effectivity of production methods, from the technologic and legal point of view. The sale price of bio-ethanol, which is being produced or will be produced from the corn or grain starch, is nearly the same as the price of purchasing raw materials. These materials are comparatively expensive but their advantage is in the easier technologically managed hydrolysis.

All above mentioned features has announced Mr. Philip W. Madson in his lecture “Bioethanol experiences in the USA” held at Lissa (Netherland) on May 1990. Finally he stated that the bio-ethanol production is on the limit of rentability, even when the technical progress continues in its development. His opinion is that new methods could solve the problem of production effectivity, namely when the government would support the legal provisions. During next several years there were issued several patents, but no one of them can solve the problem as a complex, and more economically.

The known method of bio-ethanol production has been described recently in U.S. Pat. No. 4,564,595, which has been based on the acidic hydrolysis of predelignificated cellulose, and the following fermentation of created monosaccharides (namely glucose). Most of patents describes the ethanol fermentation with lower pressure, by which the separation of ethanol from the fermentation part is going on. The separation of ethanol can be accelerated by the bubling through carbon oxide. A disadvantage of this method may be the necessary delignification of ligno-cellulosic material, and low concentration of fermentable saccharides.

There is known European patent No. 0 101 190 named “Process for producing ethanol” of two authors Mr. Assarson and Mr. Nagasuy, who use the acidic parcial hydrolysis of starch for glucose production, and the glucose is then fermented, so that the ethanol is created. As the entering raw material they consider carbo-hydratic material adjusted by various methods (chemically modificated, derived, unmodificated and/or their mixtures). The cellulose theoretically belongs among some of these groups even when the authors do not mention it on their list of raw materials. But designed conditions of hydrolysis, mainly the temperature, cut out the cellulose from the list of usable raw materials. There is possible that during the temperature 167° C., the hydrolysis of pentosan part can occure only, the ligno-cellulosic complex keeps intacted. This is the reason why only amylaceous raw material is stated on the list.

Japanese patent No. 59048090 named “Preparation of fuel alcohol” is trying to remove the high energetic demands of known methods. It is based on the fact that the monosaccharides can be prepared from renewable raw materials with the help of fermentation, and they are fermented to ethanol in the next step. The amylaceous materials are cracked with the help of the fibre fungi of genus Aspergilus, ligno-cellulosic materials like woods are treated with the help of distillery yeast, straw and similar materials with the help of Bacillus natto. All components shall be mixed in the rate 5:3:2, and the mixture shall be submitted to the alcohol fermentation. The evident disadvantage of this method is the fact that high-molecular saccharides must be submitted to the prefermentation cracking. This method needs next three fermentation units, and this type of fermentation goes on very slowly.

Designed technologies are based on the usage of amylaceous materials (namely corn and grain) until nowadays.

Comparison and knowledges from conventional method of ethanol production based on renewable materials (grain, corn) supports the development, and caused that the new industrial branch was established. Nowadays, nearly 9% of petrol consumption in USA can be supplied as mixture containing 10% of ethanol.

Description concerning the production expenses of individual factory for ethanol production as fuel alcohol, which is working up the grain or corn by dry mills, are nearly the same as the sale price of ethanol product. If there are compared the profit and losses of production expenses including the equipment, working powers, energy etc., the loss shall be resulted.

The future projects of ethanol production shall be able to bring better evaluation of the adjacent products, not only ethanol itself, and profitable sale of ethanol as fuel alcohol. Separation and treatment of the adjacent products with low expenses, which are contained in non-amylaceous fraction of raw material (grain, corn), is the subject of great interest.

The second strategy which is subjected to the reasonable interest nowadays, is to use the entering raw materials with lower expenses, as for example cellulose, so that to reduce total expenses of raw material, and finally the expenses of fuel alcohol production.

There were many factories in USA using the boiling systems designed for the prior working up the starch, without any regard to critical demands for bacterial control. Typical grains can contain as much as 10 million bacterial and mildew cells in one gram of raw material. This biological problem highly exceeds the limit for efficient fermentation. Many of these factories meet uncontrollable infections which caused the lowering of profit.

Continual method of ligno-cellulosic material hydrolysis is not used industrially until now. Very short reaction periods, to ensure quick heating of raw material mixture, and the heat regeneration can be very difficult problems, when there is necessary to ensure economic effectivity. Next disadvantage is the uncomplex usage of all products which are created with the help of these methods.

Very important condition of hydrolysic processes for ligno-cellulotic materials working up is the ensuring of operational continuity of production, universal equipment for various kinds of ligno-cellulotic materials, optimalization of hydrolysis process parameters, complex and the economical profitability of products created with the help of this type of hydrolysis, and their next utilization.

### SUMMARY OF THE INVENTION

Above mentioned disadvantages of todays' hydrolytic methods of monosaccharides (glucose), ethanol, furfural, furane, clean lignin, acetic-acid and formic-acid, alcohol fermentation residues and carbon oxide, are solving with the help of technologic and economic method of continual pressure hydrolysis of ligno-cellulotic materials, eventually with the help of an anorganic acid. The hydrolysis can go on according to the demands for technology either in one-stage or two-stage hydrolysis in accordance with the invention, which is based on the special technology, i.e. the ligno-cellulosic material, crushed to small particles 10 till 30 mm, shall go through the feeding endless screw press. Simultaneously, the material shall be moistened up a little by technological water in the rate of 0.3 till 10% of the entering material mass. Whole volume of material shall be heated to temperature 80 till 90° C., and material, which has been cut up and heated, shall be continually hydrolysed with the help of the technological water and vapour. If there is applied two-stage hydrolyse:

At first stage the hemicelluloses will be cracked to pentoses when the temperature reached 160 till 185° C. and pressure 0.6 till 1.0 MPa, after 8 till 10 minutes the hydrolysed suspension will be separated in the squeezing and conversion press to fluid phase containing pentoses, which shall go on to the expansion, and eventually to the next working up. Solid unreacted ligno-cellulosic phase shall be forced through the conversion press into the second stage of hydrolysis, in which the pressure hot water (temp. 200 till 240° C. and pressure 1.6 till 3.3 MPa, and hydro-module 1:2 till 1:3.5) shall be acting 8 till 10 minutes, and together with water the diluted solution of anorganic acid at the rate to suspension 0.1 till 1%, it shall be alternatively sprinklig. The hydrolysis is going on, whilst the solid and fluid phase is shifted as equilibrium. Phosphor-acid or hydrochlorid-acid, eventually some other acid shall be feeded by a pump, so that the treatment of pH and its acidity shall be prepared, into feeding piping in front of hydrolyser. All vapour in the hydrolytic system will be condensated up, and it will make the raw material hot. Evaporation heat will cover also the heat losses through the metal jacket of the second hydrolyser. There shall occur the hydrolytic cracking of hemicellulosis onto pentosans, and the mixture containing furfural, acetic-acid, formic-acid, methanol, hydrolytic saccharides as glucose, and the cracked ligno-cellulosic phase, which is unreacted yet.

At occasion of single-stage hydrolysis, the solution of created substances mixtures, which have the hydromodule 1:4 till 1:5 shall be led in the hydrolysers, during its movement in hydrolysers for the period 10 till 12 minutes, with the help of temperature 210 till 240° C., and the pressure 1.8 till 3.3 MPa. During this hydrolysis the cracking of pentoses onto pentosans shall be carried out, and by the dehydratation onto furfural, acetic-acid, formic-acid, methanol, and the cracking of ligno-cellulosic complex structures onto the hydrolytic saccharides shall be done. This suspension shall go on between the hydrolysers cross the overflow pressure pipe into the next hydrolytic section to be subjected to the final hydrolysis, and two or three phases of expansion to the atmospheric pressure shall follow, what will be the cause of evaporation of fluid solution containing furfural, acetic-acid, formic-acid, methanol and water. Part of vapours shall be taken away with the help of inert gases and the high-pressure expander slide-valve into heat exchanger. Mixture of hydrolytic saccharides, and the unreacted cracked ligno-cellulosic phase, which is going through press equipment where the separation of the hydrolytic saccharide solution and the unreacted solid ligno-cellulosic phase occurs. This unreacted phase shall go for the enzyme hydrolysis, and it will be worked up to glucose and clean lignin.

Next advantageously solved technological cycle of continual hydrolysis, which belongs to equipment according to the invention, is the furfural separation cycle. Vapour mixture containing water, fural, methanol, acetic-acid and formic-acid shall go from the hydrolytic cycle into the separation cycle. The mixture shall be continually feeding into the rectification column. From the rectifier it shall go away as the distilled mixture containing furfural, methanol and water, and the mixture of acids and water as distillating rest. The distillate shall be taken away into decantation vessel after its cooling, and in this vessel shall be separated as the heterogenic mixture to two layers. Top layer containing about 8 wt. % of furfural shall be returned into decantation vessel. The bottom layer containing 92 wt. % of furfural shall be kept in the supply tank. Both mixtures shall be separated by the rectification distillation in the column. Methanol which is created as the distillate shall be taken away and supplied in supply tank, and the mixture of furfural, methanol and water as distilling rest shall go away, water shall be turned back to supply tank.

During separating of mixture from the supply tank, which contains first of all furfural, the water contaminated by furfural and methanol shall be taken away from rectifier as distillate. This flow shall be taken back into the decanter. Fural in concentration of 99.67% shall be taken as distillation rest to the supply tank. In bottom part of rectifier are deposited the heavy distilling fractions according to the quality of the worked up raw material located in the hydrolytic unit. These pitches shall be time to time remove from the column, i.e. they shall be taken away to supply tank.

At occation of the direct production of furan from furfural, which may reach the volume concentration 99.67%, without simultaneous extraction of furfurylalcohol, fural shall be charged into the autoclave where shall be added catalyst (CaO, CaCO₃, MnCrO₂, or ZnCrO₂). After covering the autoclave shall be heated to temperature 400° C. There will be separated furan, which shall be cooled in heat exchanger, and it will be deposited in tank. Also carbon oxide shall be taken away from autoclave, and it is liquidated by oxidizing combustion in the combustion chamber. Production process of furan gaining will not be carrying out as continual process, because there will go the reaction in the autoclave at high temperature and pressure.

Distilling rest from the rectifier containing acetic-acid, formic-acid and water shall be taken away to supply tank and from that shall be continually sprinkled into top part of extraction column. Ethylacetate with water which can extract the acids in water shall be transported from the supply tank to the bottom part of column. Two flows shall be taken away from the extraction column. Top flow contains the water solution of acids and ethylacetate which shall be transported onto rectifier, where the water solution of ethylacetate shall be separated from the mixture, and it shall go to supply tank and there shall be recycled. Distilling rest contains the water solution of acids and rest of ethylacetate, and it shall be taken away into supply tank. Bottom flow from the extracting column, containing the rest amount of acids and ethylacetate diluted in water, shall be taken away into the supply tank. Intermediate products in the supply tanks shall be worked up as follows: both mixtures shall be entering into rectifier alternatively. Water as the distillate with low volume of ethylacetate shall be taken away at the occasion of the supply tank mixture working up. This mixture shall be sprayed again into the rectifier. Mixture of clean acids as the distilling rest shall be taken away from the column, and it shall go to the supply tank. Solution of the hydrolysate containing hydrolytic saccharides, the solid ligno-cellulosic phase as fibrous material and water shall be continually taken away from the bottom part of expander, cross a rotating shutter. From the supply tank where the solution is mixing, it is pumping onto the filtration press or centrifugal separator. After pressing there shall be gained the solution of sugar and water, and the solid fibrous ligno-cellulosic rests, which shall turned back into thermo-pressure hydrolysis for the final hydrolysis, or these rests shall be hydrolysed by the enzymes cellobiose and cellulast. These combined process provisions will ensure the high profitability of fermented sugars.

Solution of saccharides from the hydrolysis shall be continually led into the amylaceous material and the mixture shall be subjected to the amylotic hydrolysis, and the rest solid parts containing non-starch grain composition will be separated from resulted reacting mixture. Rest solid parts shall be turned back to the thermo-pressure hydrolysis. Glucose solution, first of all treated for certain pH factor, after addition of salt and nutritions, and after treatment of glucose concentration eventual dilution with unthickened distillery slops from the vapour column, cross heat exchanger, into the fermentor. New fermentation can be run very quickly as the separated yeast shall be turned back to the feeding fermentation, or 20 till 30% of fermentor volume may be kept in the fermentor as the starter of fermentation. The solution created by glucose fermentation when it will be changed to ethanol, and after the yeast separation, shall be pumped onto distillation. About 90% of ethanol in concentration nearly 40% shall be taken away in the form of vapour from distillation column into rectificating column, and the part of distillery slops shall be turned back into the fermentation, where it will dilute the saccharides solution to needed concentration, and unused distillery slops shall go on to the evaporator. Distillery slops can be thickened to the required concentration of dry substance. This whole process has in industrial conditions high profitability, and distillation effectivity is approximately 99.5%.

Compactability and linkability of all working up steps, i.e. hydrolysis, fermentation, distillation and rectification, and together with steps for the usage of side products as fural, lignin, distillation slops, yeasts and carbon oxide can ensure the automatization of production process, and reach the favourable system of renewable sources working up without any waste.

Temperature of solution entering from the thermo-pressure hydrolysis is using for the starch gelation which is being added into the solution, with eventual final heating of technological water or vapour. After the starch changes to starch jelly and adjusting of optimal water temperature suitable for the thermo-stable amylases, the process of starch cracking to glucose is going very quickly. The advantage of this method is, that the heat of hydrolytic solution can be used.

Heat of glucose solution, and heat of distillation slops can be used for preheating of water spray into vapour column. Heat of exhausting water shall be used for the improvement of energetic balance of thermo-pressure hydrolysis. Waste heat can be used in working sets very effectively. Waste water, in whole measure, shall be turned back into process, except water in moistured materials and washing water. Low concentrated acid shall be used for hydrolysis process in volume of 0.3 till 1% of mass. Very useful can be the usage of phosphor-acid, of which salts serve during the fermentation as the nutrients.

Advantage of this method and equipment is first of all the fact that the hydrolytic fermentation process is working up the entering material completely to the saleable products as fural, furylalcohol, furane, lignin, ethanol, acetic-acid, formic-acid, carbon oxide and distillery slops with yeasts.

Next advantage of the production of all above mentioned products is the solution into a compact production unit, and this method is using the ligno-cellulosic and amylaceous materials, and the agricultural vegetable can be worked up completely in wasteless process. Waste waters are the only waste.

Technical and technological core of the equipment is the complex of feeding, pressuring, conversion and transmittal machinery, and hydrolytic, decompressing and other equipments which enable the transport of entering raw materials and suspenses with the help of continually flowing and mixing operation in hydrolysers, and with the help of needed range of temperature, pressure and delay period in the hydrolytic part of ligno-cellulosic materials, where the starch is working up in the continually linkage. Process of the hydrolytic fermentation technology of bio-ethanol production has very important feature for the protection of environment, i.e. this production is solved by a compact production unit, working up the renewable sources of materials, and without any significant waste.

The equipment for this production method can be completely produced in machinery factories of the middle production volume without any dependence on the import. This method establishment enables the production of bio-ethanol and several other products from saccharidic sources, i.e. ligno-cellulosic materials, with high effectivity 75 till 85%, and with other advantages as follows:
revitalization of agricultural enterprises, more working occasions
usage of bio-ethanol which has been produced by designed technology
economical effectivity
ability for competition all over the world
reducing of imported mineral fuels (oil)
technologic universality
no harmful impact to environments
utilization of renewable sources
variability of sources
possibility of new technologies and know-how export

### BRIEF DESCRIPTION OF THE DRAWINGS

The invention shall be more detailed clarified with the help of drawings as examples where graphic description is given:

FIG. 1—block scheme of pressure and enzymatic hydrolysis, and separation

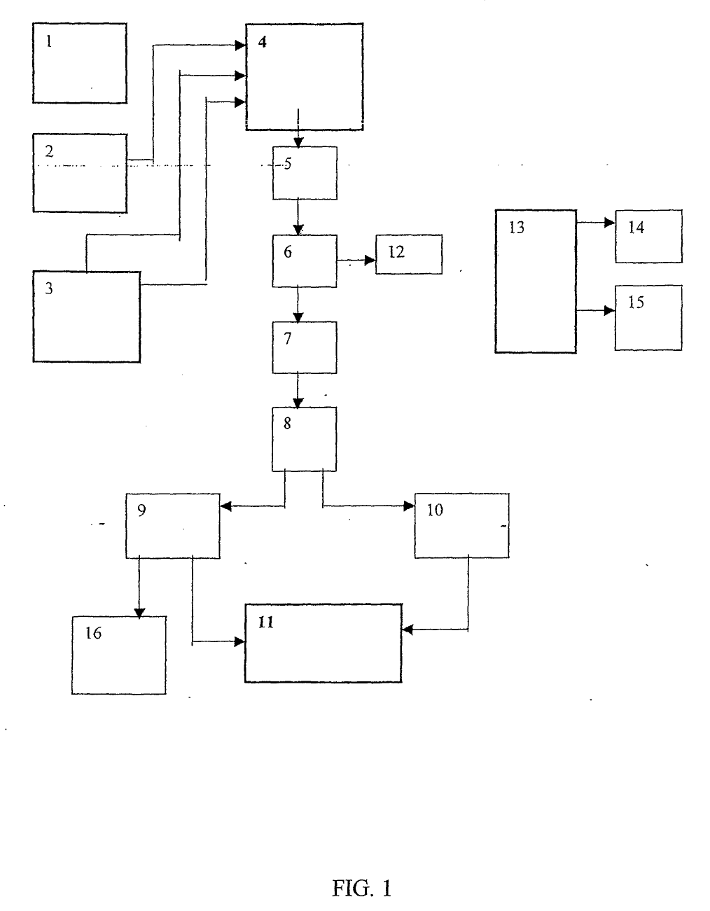

FIG. 2—block scheme of fural mixture separation from vapour phase of hydrolysis

FIG. 3—block scheme of fural working up to furane

FIG. 4—block scheme of acetic-acid and formic-acid and water separation

FIG. 5—block scheme of fermentation, and ethanol separation from glucose gained by hydrolysis of ligno-cellulosic materials and starch

FIG. 6—decompressing, conversing and transmitting endless screw into counter pressure

### DESCRIPTION OF THE PREFERRED EMBODIMENT

Ligno-cellulosic material, after its preadaptation onto small parts 10 till 30 mm, shall be compressed in the feeding endless screw press, and during this operation the material shall be moistured a little by technologic water in volume of 8% of entering material mass, which shall be compressed step by step, and simultaneously heated to the temperature of 85° C. Crushed and heated material shall be continually hydrolysed with the help of technologic water and vapour. When the two-stage hydrolysis is applied, the hemicelluloses will be cracked to pentoses in first stage at temperature 160 till 185° C. and pressure of 0.8 MPa, with the help of delay period 8 till 10 minutes and following decompressing in press, where the hydrolysed suspension shall be separated onto the fluid phase containing pentoses, and it shall go on to the expansion and eventually to next working up step. Solid unreacted ligno-cellulosic phase shall be pushed through the conversion and transmitting press into second stage of hydrolysis. Pressure hot water with temperature 220° C., and pressure of 2.8 MPa, and hydro-module 1:3 during the exposing time, i.e. delay period 8 till 10 minutes for ligno-cellulosic suspension exposition, and together with the water, the diluted solution of phosphoric-acid in the rate 0.8 to the suspension shall be feeding in two phases, and the hydrolysis shall go on with the help of simultaneous continual shift of the solid and fluid phase. Phosphoric-acid or chlorhydrogenic-acid, eventually some other acid shall be feeding with the pump for pH treatment and acidity treatment into feeding pipe in front of the hydrolyser. All vapour shall be condensing in the hydrolytic system, and the passaging material shall be heated. Condensing heat shall cover also the heat losses cross the coat of second hydrolyser. Catalytic reaction of the acid, pressure and temperature shall ensure the hydrolytic cracking of hemicelluloses to the pentosans, and the mixture containing furfural, acetic-acid, formic-acid, methanol, hydrolytic saccharides, i.e. glucose and the cracked unreacted ligno-cellulosic phase shall be created.

During one-stage hydrolysis the solution of done mixture of created substances shall be led in the hydrolyser for the delay period of 10 till 12 minutes, with the help of high temperature 210 till 240° C. and the pressure of 2.2 MPa, and hydro-module 1:5. This temperature and pressure in the acidic medium shall cause the cracking of pentoses to pentosans, and the dehydratation to furfural, acetic-acid and formic-acid, methanol, and the cracking of ligno-cellulosic complex to the hydrolytic saccharides. The suspension shall be led between both hydrolysers, cross the out-flow pressure pipe, into next hydrolytic section to the final hydrolysis. As a next step the suspension shall be kept to expand to the atmospheric pressure, and the evaporation of fluid solution part containing furfural, acetic-acid, formic-acid, methanol and water, will occur. Part of vapours shall be led away by the help of high pressure shutter into the heat exchanger. The mixture of hydrolytic saccharides and unreacted cracked ligno-cellulosic phase shall be kept in the fluid part which is transmitted from the bottom part of expander. Unreacted cracked ligno-cellulosic phase shall be pushed through the press where the separation of hydrolytic saccharides and unreacted solid ligno-cellulotic phase will occure, which shall be worked up to the glucose and clean lignin with the help of enzymatic hydrolysis, which will follow.

During the furfural separation from the hydrolytic cycle, the vapour phase containing the mixture of water, fural, methanol, acetic-acid and formic-acid is entering the separation cycle. Then the mixture shall be continually feeding into the rectification column, from where the mixture of furfural, methanol and water as the distillate, and the mixture of acids and water as distillating rest shall go away. Distillate shall be taken away into decanting vessel after its cooling, where it shall be separated as the heterogenous mixture to two layers. Top layer contains approximately 8 wt. % of furfural, and it shall be leading back into decanting vessel. Bottom layer contains 92% of furfural, and it shall be deposited in the container. Both mixtures shall be separated by rectification distillation in the column. Methanol which shall be taken away as the distillate from the tank during the separation of mixture, and it shall be deposited in a container, and the mixture of furfural and methanol with water. Water shall be turned back into container. At the occasion of mixture separating from the container, which contains mainly furfural, the water contaminated by furfural and methanol shall be taken away as the distillate from rectification column. This flow shall be led back to decanter. Furfural of concentration 99.67% shall be taken away into a container as the distillating rest. Heavy distillation fractions are deposited in the bottom part of column, according to the quality of feeding raw material in the hydrolytic line itself. These pitches shall be removed from the column time to time, i.e. they shall be taken away and deposited into the tank.

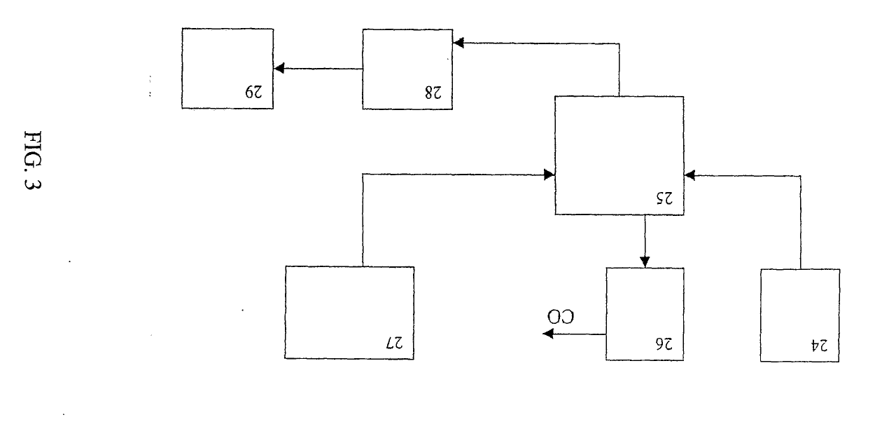

As apparent from FIG. 3, at the occation of direct furan production from furfural with the volume concentration of 99.67%, without simultaneous gaining of furfurylalcohol, the fural shall be charged from a tank into the autoclave including catalyst, in this execution example the catalyst shall be calcium oxide CaO. Autoclave shall be heated to the temperature of 400° C. after its covering. There are separated out the furan, which shall be cooled in the heat exchanger, and it shall be deposited in tank. Also carbon oxide shall go away from the autoclave which shall be liquidated by combustion in the combustion chamber. Production process of furan creating cannot be realized continually with regard to very high temperature and pressure, at which the reaction in autoclave is going on.

Distillation rest from the rectification column, containing the acetic-acid and formic-acid with water, shall be led into a container, from where it shall be continually sprayed into the top part of extraction column. Ethylacetate with water, which can extract the acids in water, shall be transported from the container into bottom part of the column. There are two flows which shall be taken away from the extraction column:

The top flow containing water solution of acids and ethyacetate which shall be transported into rectification column for separation of water solution of ethylacetate, and the ethylacetate rest, and it shall be taken away into the container, and from this container it shall be recycled. Distillation rest contains the water solution of acids, and rest of ethylacetate, and it shall be taken away into container.

The bottom flow from extraction column containing rest amount of the acids and ethylacetate diluted in water shall be taken away into another container.

Intermediate products in the containers shall be worked up as follows. Both mixtures shall be entering alternatively into the rectification column. The water with low volume of ethyacetate shall be taken away from the column at the occasion of working up of mixture from the container. This mixture shall be sprayed into the rectification column. Mixture of clean acids from the column shall be taken away as the distillation rest, and it shall go into container.

Solution of hydrolysate containing hydrolytic saccharides, defibered solid ligno-cellulotic phase and water shall be continually taken away from the bottom part of expanders, cross rotary shutter, into the moving and mixing container, from where it shall be pumped onto the fitration press or centrifuge. There can be gained the solution of saccharides and water, and solid defibered ligno-cellulotic rests, which shall be turned back to the thermo-pressure hydrolysis towards the final hydrolysis, or the enzymes shall be hydrolysed by celobiose and cellulast. These very easy combinable processing provisions can ensure high efficiency of the fermentable saccharides production.

Solution of saccharides from the hydrolysis shall be continually led into amylaceous material, and both parts shall be subjected to the amylolytic hydrolysis together, and at this occasion the solid parts containing non-amylaceous rate of grains shall be separated, and these parts shall be turned back into the thermo-pressure hydrolysis. Solution of glucose, after the treatment of pH and addition of salts and nutritions, and after the regulation of glucose concentration by eventual dilution with the unthickened distillery slops from the mash column, shall be fed cross the heat exchanger into fermentation column. Feeding fermentation with turning back of separated yields may be very suitable, otherwise 20 till 30% of the fermentor volume can be kept in fermenting column, as the starter for next fermentation, and the next fermentation will go ahead very quickly as well. Solution from the fermentor shall be pumped, after glucose fermentation to ethanol, and after yields separation, onto the distillation. Approximately 90% of ethanol with concentration about 40% shall go away as vapours into the rectification column, and at this occasion the part of distillery slops shall be turned back into fermentation, where the saccharide solution shall be diluted to needed concentration, and the unused part of distillery slops shall go on onto evapouring unit. Distillery slops can be thickened in evaporator to the required dry matter concentration. Whole process has high efficiency in the industrial conditions, and its distilling effectivity can be approximately 99.5%.

For example the wheat straw at the occasion of single-stage method, after its treatment and crushing to the particles of size 10 till 20 mm, shall be compressed in feeding press, and the material shall be simultaneously moistured by technological water with temperature of 30° C., the amount of which shall be 8% of entering mass. Raw material shall be hydrolysed with the help of temperature 220° C. and pressure 1.6 MPa, and during the period 10 minutes in two hydrolysers, the hydromodule shall be 1:4, and the hydrolysis is passing at simultaneous advancement of solid and fluid phase. After finishing of the hydrolysis, material shall be expanded in two stages, and the vapour phase and hydrolysate origins. Vapour phase contains furfural, methanol and lower organic acids, the hydrolasate contains hydrolytic saccharides,

lignin and water. Vapour phase shall be subjected to the rectification and there it is separated to the furfural mixture and mixture of acetic-acid, formic-acid and water. The hydrolysate shall be taken off the hydrolytic saccharides solution, and the unreacted solid ligno-cellulotic phase shall be led to the enzyme hydrolysis at which the degradation of solid phase to glucose occurs, and the separation of clean lignin, which shall be taken away to a container. Glucose from the first stage, and from the enzyme hydrolysis and starch shall go ahead into common container of saccharides to the preparation of fermenting process, next to the distillation and to dewatering of ethanol.

In the case of rape straw the whole process shall be the same, but the difference refers to parameters during working up: temperature 230° C. and pressure 2.3 MPa, delay period 12 minutes in the hydrolysers, the hydromodule shall be 1:4.5.

Equipment for the implementation of this method consists from equipment for the entering raw material preparation, containers, feeding pressure equipment, and at least one hydrolyser, where the last one is interconnected cross the middle-pressure expander and low-pressure expander with the moving and mixing container of hydrolytic product, and the top part of middle-pressure expander and low-pressure expander is interconnected with the top part of rectification column of furfural and with the furfural container. The equipment further contains the fermenting vessels, and the distillating column for prefermented mash containing ethanol, and the two-stage hydrolysis has between first and second hydrolysers the decompressing, conversing and transmitting press, which keeps different pressures between both hydrolysers. Single-stage hydrolysis has the outflow pressure pipe between the individual hydrolysers, and this pipe keeps the same temperatures and pressures in hydrolysers.

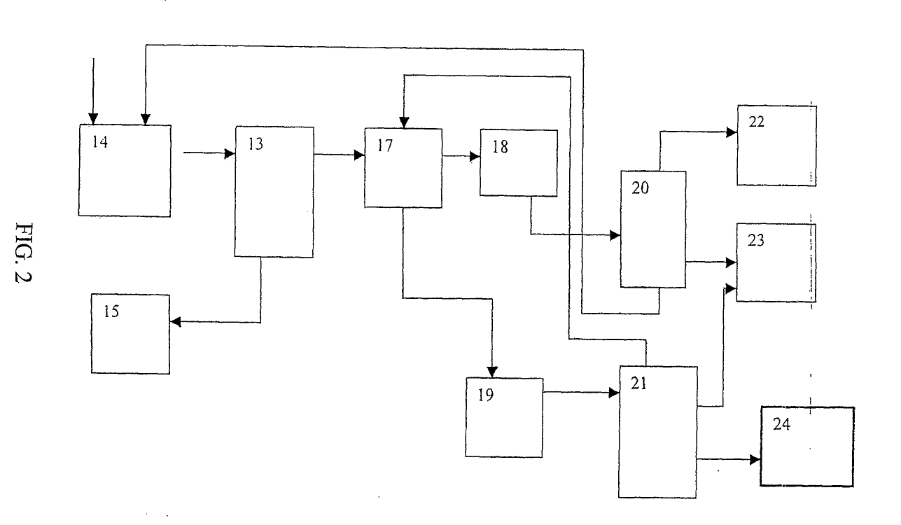

As apparent from FIG. 2 the container of furfural mixture 14 is connected to the rectification column 13, which is connected with the decanter by piping for methanol and water 17, the decanter is connected by pipe with the container for the low-percentage furfural mixed with water 18, and with the furfural container 19. Container for low-percentage furfural with water 18 is connected with the methanol column 20, and this column is connected to the methanol container 22 and 23, bottom part of the methanol column 20 is connected with the furfural mixture container 14. Furfural container 19 is connected with the vacuum rectification column 21, its top part is connected to decanter 17, and its bottom part is connected with the clean furfural container (of volume concentration 99.67%) 24, the rectification column is further connected with the methanol container 23.

Equipment for the working up of furfural to furane, as apparent from FIG. 3, contains the clean furfural container 24, which is connected with the pressure melting furnace 25, the container of catalyst 27 is connected to the furnace, and it is also connected with the oxidizing furnace by piping for carbon oxide 26. Bottom part of the pressure melting furnace 25 is connected, cross an intermediate container 28, with the container for furan 29.

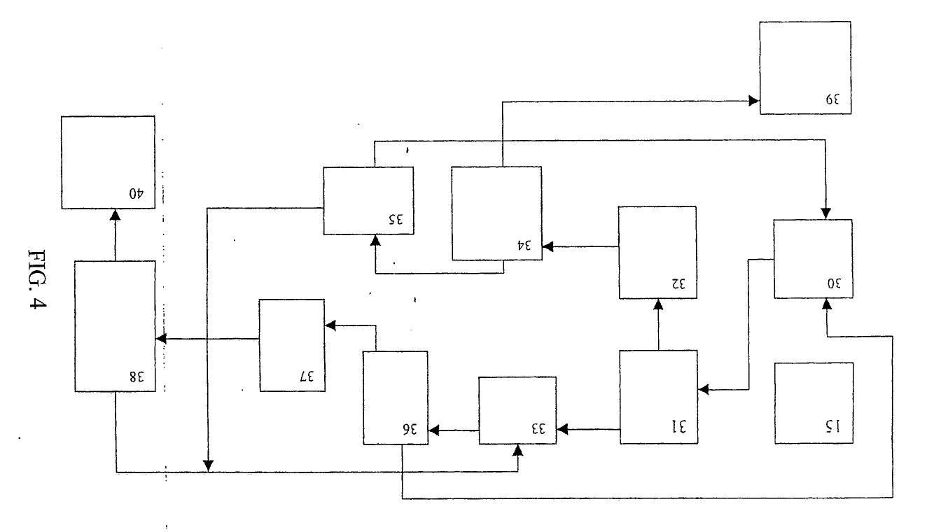

Equipment for the acetic-acid, formic-acid and water separation as apparent from FIG. 4, is combined by the acids container 15 and container for ethylacetate 30, which are connected to the extraction column. Outflow pipe from this extraction column 31 is led into the waste water container 32, this container is connected with the rectification column of waste water 34, its bottom part is connected with the waste water 39, its top part is connected with decanter 35, which is connected with the ethylacetate container 30. Rectification column 31 is connected with the container of acid, ethylacetat and water mixture 33, which is connected with the rectification column of ethylacetate 36, its top part is connected with the container for ethylactate 30, and its bottom part is connected with the container of acids 37, this one is connected to the rectification column of acids 38, its top part is connected with the acid mixture container 33, and bottom part is connected with the clean acids container 40.

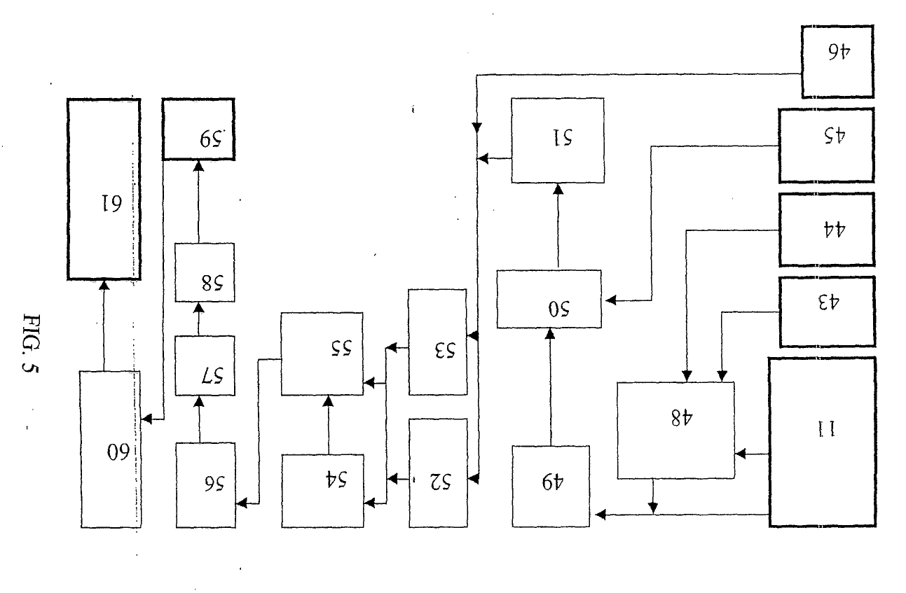

Equipment for ethanol fermentation and separation, as apparent from FIG. 5 is assembled from the saccharide solution container 11 creating in thermo-pressure and enzyme hydrolysis, which is directly connected to the heated liquefying tank 49, and as the hot water container and as the defibered amylaceous raw material 43 is interconnected with the apparatus for suspension preparation 48. This apparatus 48 is connected with the heated liquefying tank 49 which is connected with the saccharification tank 50, which is further connected with the tank for the preparation of amylolytic enzymes 45 and with the pump of sweet mash, which is connected with the fermentors 52 and 53, which are connected with the vessel for preparation of seed yeast (starter) 46 and with the yeast separation vessel 54 and feeding pump 55 of the slime pulp column 57, which is connected cross the slime pulp column cooler 57 and the crude ethanol cooler 58 with the crude ethanol container 59, which is interconnected, cross the rectification column and dewatering equipment 60, with the waterless ethanol container 61.

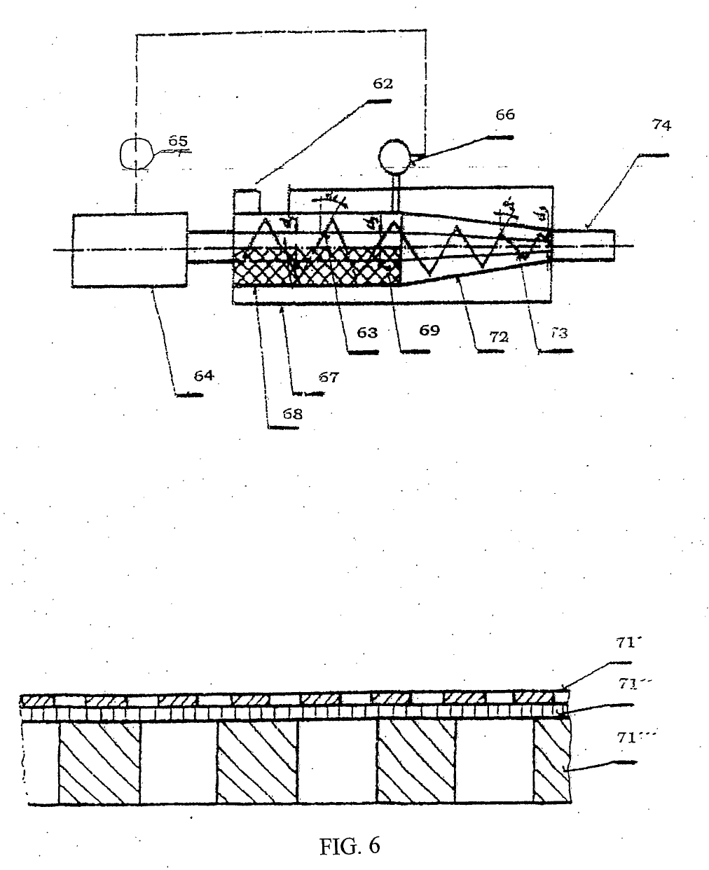

Equipment of the decompressing, conversing and transmitting press into counter-pressure, graphically descripted in FIG. 6, which is used at the case of two-stage hydrolysis, compounds from four zones. First zone is made by the feeder 63, which is represented by the cylindric body 68 and cylindric spindle 69 with a constant lead of spindle worm. Cylindric body 68 is provided by the sandwich perforation under the axis of revolution 71, second zone is made also by cylindric body and cylindric spindle with constant lead of worm and with the volume contraction of thread profile, and cylindric body is provided by sandwich perforation as well. Third zone is created by conical body 72 and conical spindle 73 with the decreasing lead of helix, and the conical body is furnished by the system of conical areas and small radial channels. Fourth zone is created by the pressure feeding head 74 of cylindric shape, and the driving gear unit 64 with transmission interconnected with the spindle. Driving gear unit is provided by a switching ampermeter 65 eventually interconnected with the fluid feeding equipment 66.

### FIELD OF THE APPLICATION

### The Invention can be Utilized

The main product of continual production process is ethanol of concentration more than 98% of mass. Several possible variants are important for its utilization:

A) Ethanol can be added into motor fuels
B) Ethanol itself can be used for the energy production
C) Other utilities

A. Variant “A” appears as the most advantageous utility of ethanol, so as additive to the motor fuels. Possibility of the direct ethanol addition in rate 10 till 30% into motor fuels without any necessary adjustment or change of motor construction. Effect of the addition may be apparent in the lowering of harmful exhaust emissions (namely CO).

B. Variant B. Ethanol itself usage for the energy production may be designed in case, if process of ethanol addition into motor fuels should be somewhere very problematic. This variant is connected with the idea of electric power or heat energy production, and according to the needs in individual region. This variant would make possible to add ethanol, industrially produced, to combustion process, what would increase the heating capacity of solid or gas fuel, for example the entering of ethanol with the help of spray nozzle into the combustion chamber with burners.

C. This variant has several partly possibilities, which are individually limited by capabilities of customers. One of this partly variant is the usage of some part of ethanol industrial production in the industry of paints and lacquers as a solvent. Another possibility is the treatment of some production part of ethanol for chemical and food industry.

Further important products and their utilization:

Further most important products of ethanol industrial production are furaldehyd, lignin, acetic-acid, formic-acid, limited amount of methanol, carbon oxide, distillery slops and yeasts.

Lignin is well salable raw material, required namely as the part of filling materials in the rubber industry, and it has very favourable features for the quality of produced materials (mainly for tyre production).

Furaldehyd, acetic-acid, and formic-acid are the commodities, which can be well sold on the chemical product markets. As lignin, all of them are well salable products, which can be produced in needed quality, and have good impact to economy of ethanol production.

Methanol is well salable on chemical product markets, as it can be reasonably used in the industry of motor fuels.

Carbon oxide (CO₂) is taken away relatively in huge amount and quality (it is practically clean exit from the biologic process fulfilling all demands of food industry). There is obvious, that its taking away can create the important part of production.

Distillery slops and yeasts can be used in the agriculture. There is suggested to use them alternatively as the raw material for biologic gas production.

### LIST OF REFERENCE SIGNS

1 Preparation of Raw Materials
2 Raw materials container
3 Heating cycle
4 Feeding equipment with hydrolysers
5 Pressure shutter
6 Expanders
7 Fluid product container
8 Press—separator of solution and solid rests
9 Enzyme hydrolysis
10 Solution of hydrolytic saccharides, i.e. glucose
11 Container for monosaccharides created at pressure and enzyme hydrolysis
12 Fural container
13 Rectification column
14 Fural container
15 Container of acids
16 Container of lignin
17 Decanter
18 Container of low-percentage fural
19 Fural container
20 Methanol column
21 Vacuum rectification column
22 Container of methanol
23 Container of methanol fraction
24 Container of clean fural of volume concentration 99.67%
25 Pressure melting furnace
26 Furnace for CO oxidation
27 Container of catalyst
28 Cooler/heat exchanger
29 Furan container
30 Ethylacetate Container
31 Extraction column
32 Waste water container
33 Container for mixture of acids, ethylacetate and water
34 Rectification column of waste water
35 Decanter
36 Rectification column of ethylacetate
37 Container of acids
38 Rectification column of acids
39 Waste water container
40 Container of clean acids
41 Containers for grain raw material
42 Tank for lime milk
43 Hammer mill for crushing of amylaceous raw material
44 Hot water tank (hot-well)
45 Enzyme tank
46 Fermenting tank
47 Pump of seed yeast (starter)
48 Preparation of Suspension
49 Heated liquefying tank
50 Saccharification tank
51 Cooler and pump of sweet mash
52 Fermenting tank—fermentor
53 Fermenting tank—fermentor
54 Yeast separation
55 Feeding pump of slime pulp
56 Slime pulp intensificating column
57 Cooler of slime pulp column
58 Cooler of crude ethanol
59 Crude ethanol container
60 Rectification and dewatering of ethanol
61 Container of waterless ethanol
62 Feeding
63 Feeder consists of cylindric body
64 Driving gear unit
65 Ampermeter
66 Injection equipment
67 Cylindric coat/channel
68 Cylindric body
69 Cylindric spindle
70 Sandwich perforation
71′ Perforated metal sheet
71″ Filtration netting
71′″ Outer perforated coat
72 Conical body
73 Conical spindle
74 Pressure feeding head

1. A method for production of monosaccharides, ethanol, furfural, furane, methanol, acetic-acid, formic-acid, and lignin produced from polymer materials by the continual thermo-pressure hydrolysis, combined with the enzyme hydrolysis, wherein polymer material, disintegrated to particles of the size 10 to 30 mm, is subjected to the continual thermo-pressure hydrolysis, at which occasion the hydrolysis can be made at single-stage or two-stage, at different technological conditions, according to the quality of entering material and the requirement for the exit products, and after the hydrolysis creating suspension shall expand to the middle-pressure and normal atmospheric pressure at least in two stages, by which it is separated to vapour phase containing besides the water also furfural, methanol, acetic-acid, formic-acid, and to fluid phase containing the water solution of hydrolytic saccharides and other dilatable matters, and to unreacted solid ligno-cellulosic phase, which is separated by pressing and/or filtration, and after addition of water they is subjected to the cellulosic enzymes acting, and at this reaction monosaccharides are formed, which are subjected to amylolytic hydrolysis after separation of uncracked lignin, together with the treatment of amylaceous materials and fluid phase, after which the saccharide solution is further worked up, and/or after which the saccharide solution as sweet mash is fermented onto ethanol, which is dehydrated and concentrated after separation of the yeast, then the disintegrated raw material is moistened by water spraying at the place of feeding pressure equipment (warm water of 20 to 40° C.) in the amount of 5 to 10% of mass of entering material.

2. The method according to claim 1, wherein the hydrolysis is a two-stage process, a first stage by the temperature of 150 to 185° C. and pressure 0.6 to 1.0 MPa and at the hydromodule 1:4, and a second stage there shall be sprayed the additional pressure warm water of temperature 200 to 240° C. and pressure of 1.6 to 3.3 MPa, and at this occasion the hydrochloric-acid or some other suitable acid is simultaneously sprayed in volume 0.1 to 1% related to the suspension with hydromodule 1:3 to 1:4, and the fluid phase is separated after finishing of the first stage of hydrolysis, and after its thickening is used as the raw material for fermentation to ethanol or furfural.

3. The method according to claim 1, wherein fluid phase from the pressure and enzyme hydrolysis is used as solution for the gelation of amylaceous raw material, and as the energetic substrate for fermentation to ethanol.

4. An apparatus for implementation of the method according to claim 1, consisting of apparatus for preparation of entering raw materials, containers, feeding pressure equipment, and at least one hydrolyser which is connected, cross the middle-pressure expander and low-pressure expander, with the moving and mixing container for hydrolytic product, and the top part of middle-pressure expander and low-pressure expander is connected with the top part of rectification column of furfural and with the furfural container, next this equipment contains the fermentation vessels and distillation column for fermented mash containing ethanol, wherein a two-stage hydrolysis shall have between first and second hydrolyser the decompressing, conversing and transmitting press, which can keep the different pressures between hydrolysers.

5. An apparatus for implementation of the method according to claim 1, containing apparatus for preparation of entering raw materials, containers, feeding pressure equipment, and at least one hydrolyser which is connected, cross the middle-pressure expander and low-pressure expander, with the moving container for hydrolytic product, and the top part of middle-pressure expander and low-pressure expander is connected with the top part of rectification column of furfural and with the furfural container, next this equipment contains the fermentation vessels and distillation column for fermented mash containing ethanol, wherein a single-stage hydrolysis shall have the pressure overflow pipe, which can keep the same temperatures and pressures in hydrolysers.

6. The apparatus according to claim 4, wherein the container for furfural mixture (14) is connected to the rectification column (13), which is connected with decanter (17) by the piping for methanol and water, the decanter is connected with container for low-percentage furfural with water (18) and with the container for furfural (19), the container for low-percentage furfural with water is connected with the methanol column (20), and this column is connected to methanol container (22) and (23), bottom part of methanol column (20) is connected with the furfural mixture container (14), furfural container (19) is connected with the vacuum rectification column (21), its top part is connected to decanter (17), and its bottom part is connected with the clean furfural (volume concentration 99.67%) container (24), the rectification column (21) is further connected with the container for methanol (23).

7. The apparatus according to claim 6, wherein the container of clean furfural (24) is connected with the pressure melting furnace (25), to which the container for catalyst (27) is connected, and with the oxidating furnace (26) connected by the piping for carbon oxide, the bottom part of the pressure melting furnace (25) is connected with the furan inter-container (28).

8. The apparatus according to claim 4, wherein the container of acids (15) and container for ethylacetate (30) are connected to the extraction column (31), the outlet from this extraction column (31) is led into the waste water container (32), which is connected with the rectification column of waste water (34), its bottom part is connected with the waste water container (39), its top part is connected with decanter (35) which is connected with the ethylacetate container (30), the rectification column shall be next connected with the container for mixture acids, ethylacetate and water (33), which is connected with the rectification column of ethylacetate (36), its top part is connected with container for ethylacetate (30), and its bottom part is connected with the container of acids (37) which is connected with the acid mixture container (33), and its bottom part is connected with the container of clean acids (40).

9. The apparatus according to claim 4, wherein the container of saccharide solution (11) as a part of thermo-pressure and enzyme hydrolysis is directly connected onto the heated liquefying tank (49), and equally as the container of warm water (44) and container of crushed amylaceous raw material (43), it is connected with the apparatus for suspension preparation, which is connected with the heated liquefying tank (49) connected with the saccharifying tank (50), which is connected with tank for the preparation of amylolytic enzymes (45) and pump of sweet mash (51), this pump is connected with the fermentors (52) and (53), connected with the vessel for starter preparation (46) and with the yeast separation equipment (54) and feeding pump (55) of slime pulp column (56), which is connected with the container of crude ethanol (59) cross the slime pulp column cooler (57) and the crude ethanol cooler (58), the crude ethanol container (59) is interconnected with the waterless ethanol container (61) cross rectification column and cross dewatering device (60).

10. The apparatus according to claim 4, wherein it can be used at the occasion of the two-stage hydrolysis, which compounds from four zones, first zone is made by the feeder (63), which is represented by the cylindric body (68) and cylindric spindle (69) with a constant lead of spindle worm, and cylindric body is provided with the sandwich perforation under the axis of revolution (71), second zone is made also by cylindric body and cylindric spindle with constant lead of worm and with the volume contraction of thread profile, and cylindric body is provided with sandwich perforation as well, third zone is created by conical body (72) and conical spindle (73) with the decreasing lead of helix, and the conical body is furnished with the system of conical areas and small radial channels, and fourth zone is created by the pressure feeding head (74) of cylindric shape, and the driving gear unit (64) with transmission interconnected with the spindle can be provided by a switching ampermeter (65), eventually interconnected with the fluid feeding equipment (66).
